(19) United States
(12) Patent Application Publication
(10) Pub. No.: US 2011/0274728 A1
(43) Pub. Date: Nov. 10, 2011

(54) Compositions and Methods for Alleviating Hyposalivation and for Providing Oral Comfort

(76) Inventors: David Hamlin, Langhorne, PA (US); Satyanarayana Majeti, Liberty Township, OH (US); Zbigniew Pietrzkowski, Aliso Viejo, CA (US)

(21) Appl. No.: 13/129,210

(22) PCT Filed: Nov. 13, 2009
(86) PCT No.: PCT/US09/64453

§ 371 Date: Jul. 1, 2011

Related U.S. Application Data
(60) Provisional application No. 61/114,359, filed on Nov. 13, 2008.

(51) Int. Cl. A61K 9/00; A61K 36/82; A61P 1/02; A61K 36/18; A61K 36/752; A61K 36/87; A61K 36/45

(52) U.S. Cl. 424/400; 424/766; 424/729; 424/732; 424/777; 424/736; 424/725

(57) ABSTRACT

Various compositions and methods are provided to alleviate one or more symptoms associated with hyposalivation or xerostomia. Preferred compositions and methods employ one or more plant extracts and/or a proanthocyanidin at a concentration effective to reduce or eliminate the symptoms. Especially preferred extracts are isolated from a fruit and/or seed (e.g., grape, cranberry, blue berry, black berry, etc.), and where a second extract is present, such extract is preferably isolated from a citrus fruit (e.g., lemon, orange, lime, grapefruit, tangerine, mandarin orange, ugli fruit, etc.).

This application claims priority to our copending U.S. provisional application having Ser. No. 61/114,359, filed Nov. 13, 2008, which is incorporated by reference herein in its entirety.

### FIELD OF THE INVENTION

The field of the invention is compositions and methods to alleviate or treat symptoms associated with hyposalivation and/or to provide oral comfort.

### BACKGROUND OF THE INVENTION

Hyposalivation and xerostomia (commonly referred to as ‘dry mouth’) are relatively common among a variety of people, and are especially common among the elderly, patients taking various medications (e.g., chemotherapeutic agents, antihistamines, antidepressants, diuretics, etc.), and individuals with high coffee or alcohol consumption. Xerostomia is also often found in patients with endocrine disorders, nutritional deficiencies, nerve damage to the face, neck, and head, and even stress. Most typically, patients diagnosed with xerostomia, including those with Sjogren's Syndrome will have a significantly reduced flow and volume of saliva, commonly also associated with chemical changes in the saliva, all of which often presents itself in a variety of symptoms, including bad breath, thick and string-like saliva, an altered sense of taste and, in some cases, difficulty speaking or swallowing. In more severe cases, xerostomia is also associates with oral infections, sores or cracked tissue along the corners of the mouth.

Non-Sjogren's or rheumatoid/inflammatory disease suffers are more likely to have intermittent discomfort associated with a noticeable paucity of saliva. Eighty percent of all instances of hyposalivation are directly attributable to the use of prescription drugs or over the counter medications known to interact with the central nervous system. However, even otherwise healthy individuals experience periods of hyposalivation, typically associated with high stress, physical exhaustion, or diurnal effects of hormones. For example, many individuals experience periods of hyposalivation while sleeping as mastication and general stimulation to the salivary glands is reduced during this time. While most individuals ignore transient hyposalivation, it is preferable to avoid it, if possible. Overall oral comfort is associated with sufficient saliva to create a feeling of moisture, lubrication, hydration of the soft tissues, and the ability to move the tongue and speak, swallow, and chew without discomfort. The average flow rates for a healthy adult is generally recognized to be about 1.5 ml per minute. When an individual's salivary output drops to about 50% of their normal flow, they are more likely to notice hyposalivation and its effects on their oral comfort.

There are many compositions and methods known for the treatment of xerostomia. For example, U.S. Pat. No. 4,980,177 teaches mechanical stimulation with a chewing gum that further includes hydrophilic compounds. Similarly, U.S. Pat. No. 4,997,654 teaches a chewing gum formulation with relatively high xylitol content to promote salivation and U.S. Pat. No. 6,656,920 teaches use of disaccharides in a composition to treat xerostomia. Alternatively, as described in U.S. Pat. No. 4,820,506, an organic acidulant and sweetener are used to promote saliva production and/or flow. Similarly, WO 89/09594 teaches use of an organic acid in a controlled release chewing gum formulation, and U.S. Pat. App. No. 2006/0204551 teaches a synergistic combination of a food acid and a tingling sensate to promote salivation. While such compositions tend to provide at least some relief to a patient, several disadvantages nevertheless remain. For example, use of chewing gum is typically not recommended over night and thus often limited to daytime use. Moreover, and especially where acids are employed, prolonged exposure may result in at least partial dental demineralization.

To avoid at least some of these problems, nutritionally acceptable and chemically defined compounds may be administered as described in U.S. Pat. App. No. 2007/0128284 where a sulfur-containing antioxidant such as N-acetylcysteine is combined with a polymeric base, or in U.S. Pat. App. No. 2004/0076695 where omega-3 fatty acids are used in various compositions. In yet further known compositions, peroxidized lipids (typically plant oils) and silica are used to alleviate xerostomia as taught in U.S. Pat. App. No. 2006/0078620. In yet other known methods, glycerol may be employed to improve dry mouth conditions as noted in U.S. Pat. App. No. 2009/0263467A1. Still further known compositions include those in which certain plant extracts are used to formulate a composition for treatment of xerostomia as described in U.S. Pat. No. 4,938,963 (Yerba Santa extract) and U.S. Pat. No. 6,746,697 (Heliopsis Longipes extract). Yet further known compositions and methods are described in WO 2007/092811.

While at least some of these compositions will provide temporary relief in a patient, several disadvantages nevertheless remain. For example, the chemical stability of some of the compounds (and even some of the plant extracts) may be problematic. Moreover, and depending on the particular formulation, the obtained effect is relatively weak and thus requires repeated administration and/or high concentration of the active ingredient, which may be prohibitive due to bad taste or solubility.

Therefore, while numerous compositions and methods to reduce symptoms of xerostomia are known in the art, there is still a need to provide improved compositions and methods for alleviation of symptoms associated with xerostomia, and promote overall oral comfort/feel in the general population.

### SUMMARY OF THE INVENTION

According to the present invention compositions and methods are provided to reduce or even eliminate one or more of the symptoms associated with hyposalivation or xerostomia (which may be drug-induced, due to Sjogren's Syndrome, age, or other condition) wherein especially preferred compositions and methods will employ one, and most preferably at least two plant extracts. One of the extracts is most preferably isolated from a fruit and/or seed (e.g., grape, cranberry, blue berry, black berry, etc.) and the other isolated from a citrus fruit (e.g., lemon, orange, lime, grapefruit, tangerine, mandarin orange, ugli fruit, etc.). In further especially preferred aspects, the two extracts are a synergistic combination of extracts (e.g., grape seed extract and lemon extract).

Viewed from another perspective, it is contemplated that the compositions according to the inventive subject matter will comprise a (preferably naturally occurring) proanthocyanidin at a concentration effective to reduce at least one symptom associated with hyposalivation or xerostomia.

Therefore, in one aspect of the inventive subject matter, a method of providing relief from one or more symptoms (e.g., dry mouth, bad breath, thickened saliva, altered sense of taste, difficulty speaking or swallowing) that are associated with hyposalivation in which in one step a topical composition for oral administration is formulated, wherein the composition includes a proanthocyanidin and/or a grape seed extract at a concentration effective to provide relief from the symptom. In another step, a test result is obtained that indicates that the proanthocyanidin and/or the grape seed extract provide relief from the symptom, and in yet another step, the composition is provided to a consumer in association with the test result.

In most preferred aspects, the topical formulation is a mouth rinse, a toothpaste, a dissolving strip, or a lozenge, and/or the proanthocyanidin is incorporated into the topical formulation as part of a plant extract (typically as part of a bilberry extract, cranberry extract, black currant extract, black tea extract, and/or chokeberry extract). While numerous concentrations for the proanthocyanidin and/or a grape seed extract are contemplated, particularly suitable concentrations are relatively low and will typically be at or below 1 wt % of the topical composition.

It is further generally preferred that the topical composition further comprises an organic acid or an extract from a citrus fruit (e.g., lemon extract), which is most preferably present in a synergistic amount. With respect to the test result it is preferred that the test result demonstrates in vitro M3 receptor agonist activity of the proanthocyanidin and/or the grape seed extract, or that the test result demonstrates an in vivo increase in salivary flow in a human.

Viewed from a different perspective, the inventors therefore also contemplate a an oral care product to provide oral comfort to a person affected by a symptom of hyposalivation (e.g., dry mouth, bad breath, thickened saliva, altered sense of taste, and difficulty speaking or swallowing). Most preferably, the product comprises a composition that includes as sialagogue active ingredient a proanthocyanidin, a grape seed extract, and/or a plant extract that is demonstrated to have M3 receptor agonist activity at a concentration effective to alleviate a symptom associated with hyposalivation. Preferably, the composition is formulated for oral topical administration.

As noted above, it is generally preferred that the oral care product is formulated as a mouth rinse, a toothpaste, a dissolving strip, gel, gel in a tray, spray, non-dissolving strip, patch, bandage, or a lozenge, and/or that the proanthocyanidin is incorporated into the composition as part of an extract selected from the group consisting of a bilberry extract, a cranberry extract, a black currant extract, a black tea extract, and a chokeberry extract. Regardless of the source, it is further preferred that the proanthocyanidin and/or the grape seed extract are present at a concentration of equal or less than 1 wt % of the topical composition. Where desired, the composition may further include an organic acid or an extract from a citrus fruit.

Various objects, features, aspects and advantages of the present invention will become more apparent from the following detailed description of preferred embodiments of the invention.

### BRIEF DESCRIPTION OF THE DRAWING

FIG. 1 is a graph depicting saliva production (by weight) in vivo before and after topical application of selected extracts according to the inventive subject matter.

FIG. 2 is a graph depicting saliva production (by volume) in vivo before and after topical application of selected extracts according to the inventive subject matter.

FIG. 3 is a graph depicting saliva pH in vivo before and after topical application of selected extracts according to the inventive subject matter.

FIG. 4 is a graph depicting dose-response results for M3 receptor stimulation using selected extracts in vitro according to the inventive subject matter.

### DETAILED DESCRIPTION

The inventors have discovered that oral comfort can be provided to an individual, and particularly to an individual suffering from one or more symptoms associated with Sjogren's syndrome, xerostomia, or hyposalivation by administering a composition that comprises a grape seed extract, a proanthocyanidin, and/or a plant extract that includes a component that activates the M3 receptor in the salivary glands. In preferred aspects of the inventive subject matter, the proanthocyanidin is provided as part of a plant extract, and optionally a citrus fruit extract and/or organic acid is added to the composition. Most typically, the composition is formulated for oral topical administration to a mucosal membrane.

It should be noted that the term “hyposalivation” as used herein generically refers to a reduction in saliva production, flow, and/or volume as compared to normal saliva production, flow and/or volume in a healthy person. The term “xerostomia” as used herein refers to one specific manifestation of hyposalivation, which may be due to various causes. For example, xerostomia may be due to medication, radiation treatment, or an autoimmune disease (e.g., Sjogren's syndrome). It should also be appreciated that while contemplated compositions and methods are particularly advantageous for treatment of hyposalivation, otherwise healthy people can also experience occasional dry mouth during waking hours as well as during the night due to decreased salivary flow. The use of contemplated methods and compositions before going to bed will help such population sleep better and wake up with fresher mouth feel and breath, and will also provide increased oral comfort.

In an especially preferred aspect, a composition for relief from a symptom associated with hyposalivation includes a combination of a grape seed extract and a lemon extract. It should be noted that the term “grape seed extract” as used herein expressly excludes grape seed oil. Viewed from a different perspective, grape seed extract as used herein represents the fraction of grape seeds that is extracted using polar and/or hydrophilic (water miscible without phase separation) solvents. In contrast, grape seed oil is the substantially water-immiscible lipid phase of grape seeds, typically comprised of linoleic acid, oleic acid, palmitic and stearic acid. Thus, grape seed oil and grape seed extract are mutually exclusive.

For example, in one especially preferred aspect of the inventive subject matter, a topical composition was prepared that included commercially available grape seed extract (N31) and lemon extract (N600) (both obtained from VDF Futureceuticals, 819 N. Dixie Hwy., Momence, Ill. 60954) in proportions as indicated in Table 1 below with numbers indicating wt % fraction of the formulation.

TABLE 1

| | N31 | N600 |
|---|---|---|
| | (GSE) | (Lemon) |
| Formula 50 | 0.5 | 4.5 |
| Formula 10 | 0.1 | 0.9 |
| Formula 5 | 0.05 | 0.45 |
| Formula 1 | 0.01 | 0.09 |

Using the formulation labeled “Formula 10”, six subjects known to suffer from drug induced dry mouth were treated as follows: 1 mg of powder was spread on a single layer of woven cotton gauze and placed against the parotid glands on each inside side of the cheek for 5 minute exposures. Thus, total exposure was 2 mg of the topical formulation, comprising a total of 2 microgram (mcg) of grape seed extract. The results were quantitated using resting saliva collection, and humidity of tissues (cheek pouch) taken before and after the exposure as indicators. Post hoc analysis also included measurement of pH and buffering capacity of pre- and post-resting saliva samples.

Remarkably, and despite the apparent small quantity of the extracts/proanthocyanidin in the extract, apparent uniform improvement was achieved in saliva flow, saliva volume, and saliva weight (each typically having an increase of at least 10%, more typically at least 25%, even more typically at least 50%, and most typically at least 75%), which was accompanied with an increased pH indicating fresh saliva production. It should be noted that increased pH is especially advantageous as such increase reduces the likelihood of dental demineralization. Subject reactions to trial were immediate and appreciated as improved oral conditions were noted and voiced by all.

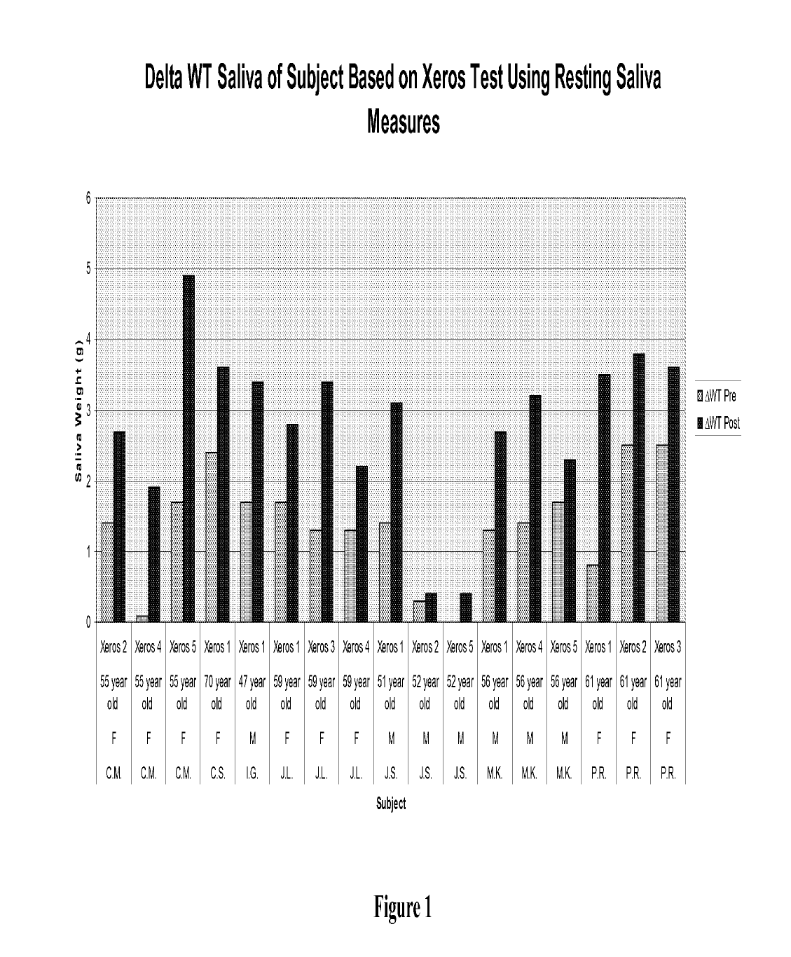

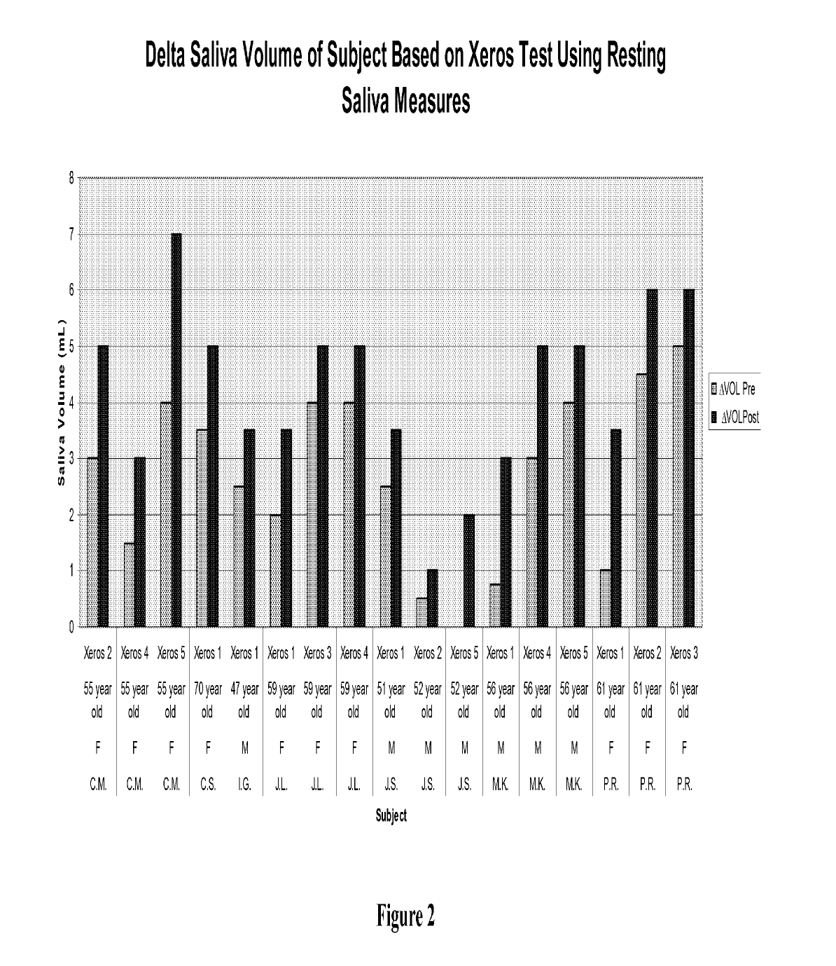

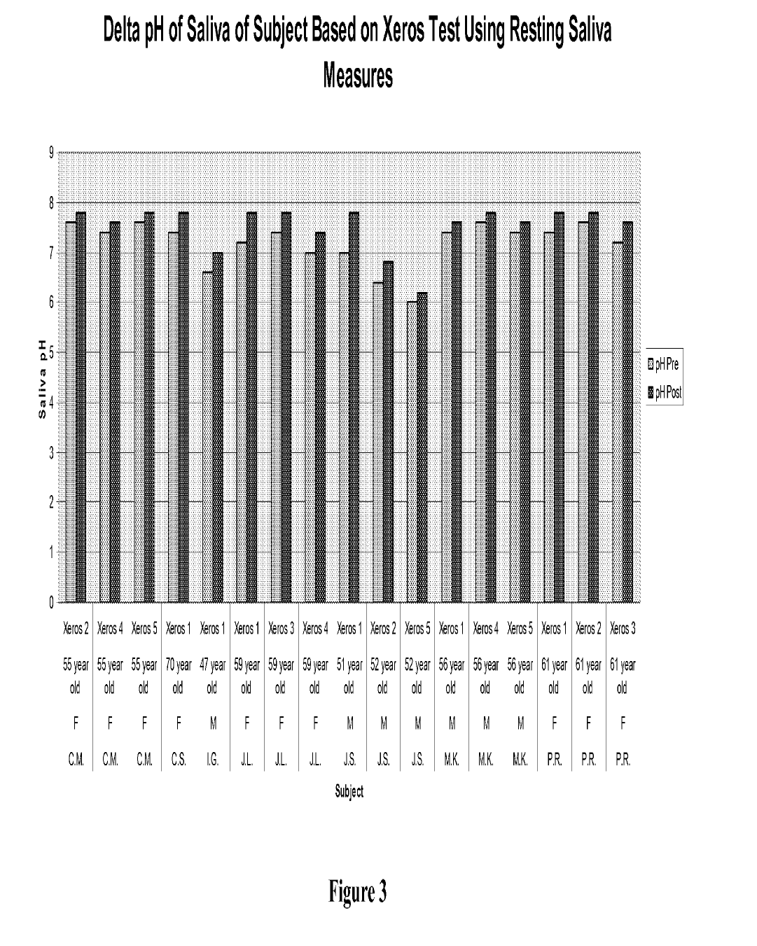

Further experiments were conducted with additional compositions and combinations (only selected data provided here), and the results depicting increased weight of saliva are depicted in FIG. 1, while the results depicting increased volume of saliva are depicted in FIG. 2. The difference in pH of the saliva before and after treatment is shown in FIG. 3. More specifically, and with further reference to the FIGS. 1-3, five experimental conditions were tested and labeled as Xeros 1-5: Xeros 1 included a pre-rinse with 30 mL water for 60 sec., followed by application of DMRF10-powder on a 2 cm×2 cm 1 ply non-woven sponge. Xeros 2 included a pre-rinse with 30 mL water for 60 sec., followed by application of Formula 50 (F-50) on a 2 cm×2 cm 1 ply non-woven sponge with powder; Xeros 3 included a pre-rinse with 30 mL water for 60 sec., followed by application of 100 mg Formula 10 (F-10) in 30 mL of water for 60 sec; Xeros 4 included a pre-rinse with 30 mL water for 60 sec., followed by application of 200 mg Formula 50 (F-50) in 30 mL water for 60 sec; Xeros 5 included a pre-rinse with 30 mL water for 60 sec. (5 min collection time), followed by rinsing with Formula 50 (F-50) in 30 mL for 60 sec (5 min collection time). All probands reported that the compositions tasted good, worked fast, and provided long-lasting relief. Indeed, all probands expressed interest to continue treatment at home as the treatment significantly exceeded expectations. Continued use appeared to provide general improvement over time.

Among other beneficial effects, individuals affected with hyposalivation reported reduction and in some cases even total resolution of dry mouth, bad breath, thickened saliva, altered sense of taste, and/or difficulty speaking or swallowing. Moreover, and especially where contemplated formulations are administered over a longer period, it should be noted that due to the strong anti-oxidant character of proanthocyanidins (especially polyphenolic proanthocyanidins), various additional benefits may be obtained, including reduction in plaque, gingivitis, and periodontal disease.

Moreover, it should be appreciated that in several experiments, and especially in certain in vitro experiments on M3 stimulation (data not shown), the inventors observed that where a grape seed extract and lemon extract were used, synergistic stimulation of the M3 receptor. For example, where the grape seed extract and lemon extract were used at a wt ratio of 9:1, activation levels of 24% were achieved, while the combined expected level of the extracts alone would have been 18% (12+6%). Remarkably, when the lemon extract was combined with a green tea extract in the same ratio, the effect was merely additive (18%). Thus, particularly preferred combinations are those in which grape seed extract is combined with lemon extract, and where the weight ratio of the extracts is between 25:1 and 2:1, more preferably between 15:1 and 4:1, and most preferably between 12:1 and 5:1.

Thus, it is contemplated that a method of providing relief from a symptom associated with hyposalivation includes a step of formulating a (most preferably topical) composition for oral administration that includes at least one of a proanthocyanidin and a grape seed extract at a concentration effective to provide relief from the symptom. It is generally preferred to obtain a test result that indicates that the proanthocyanidin and/or grape seed extract provides relief from the symptom. In especially preferred methods, the composition is made available to a user or wholesale entity or retailer together with the test result (as validation or marketing tool).

While not wishing to be bound by any specific theory or hypothesis, the inventors contemplate that the fruit/seed extract/proanthocyanidins as well as the citrus extract act as M3 receptor (Type 3 muscarinic acetylcholine receptor) agonists, most likely in a synergistic manner. Therefore, it should also be recognized that all plant extracts that activate the M3 receptor are deemed suitable for use herein as standalone compound, synergistic additive, and/or adjuvant.

Viewed from a different perspective, an oral care product is contemplated that will provide oral comfort to a person affected by one or more symptoms of hyposalivation. Especially contemplated care products include a composition that includes a grape seed extract, a proanthocyanidin, and/or a plant extract that is demonstrated to have M3 receptor agonist activity as sialagogue active ingredient at a concentration effective to alleviate at least one symptom associated with hyposalivation, wherein the composition is formulated for oral topical administration. It should be noted that the term “sialagogue active ingredient” when used in conjunction with the grape seed extract, a proanthocyanidin, and/or a plant extract that is demonstrated to have M3 receptor agonist activity means that the grape seed extract, the proanthocyanidin, and/or the plant extract are the active ingredients that promote salivary production and flow. Moreover, the term “oral topical administration” of a composition or formulation refers to contacting the oral tissues with the composition or formulation over a period that is significantly longer that the contact time typically observed for ingestion of the composition or formulation. While any ingested (swallowed) composition or formulation will at least in part contact oral tissues, oral administration of a composition or formulation for the purpose of ingesting the composition or formulation is expressly excluded from the scope of the definition provided herein. Viewed from a different perspective, oral topical administration of a composition or formulation will achieve the intended purpose (e.g., increase in salivary flow or volume) by contact with the oral tissue and without ingestion of the composition or formulation.

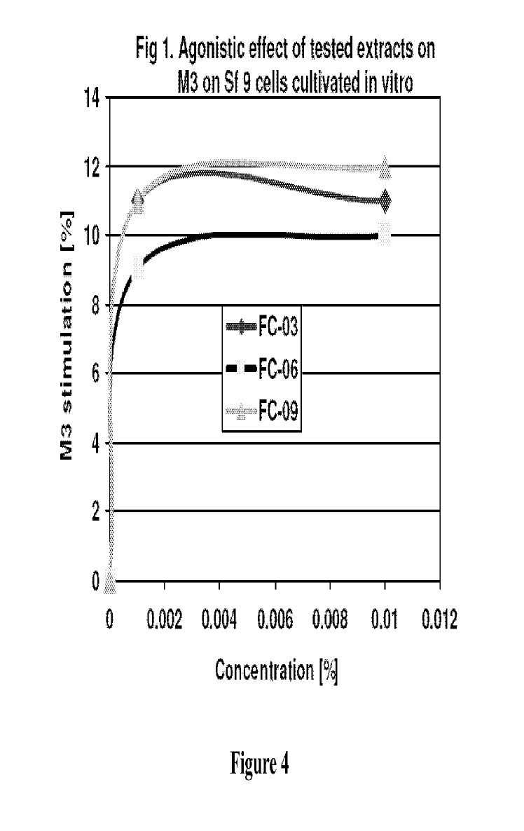

Regardless of the particular ingredients and formulation, it should be noted that the increase in saliva production, flow, and/or volume, and/or increase in M3 activity using contemplated compositions may be verified in numerous manners well known in the art. For example, M3 agonist activity can be measured using a commercially available test service (e.g., test #252810 from MDS Pharma Sciences, 2200 Renaissance Blvd., Suite 400, King of Prussia, Pa. 19406) or in recombinant cells or cells obtained from a mammal substantially following a test protocol as described in Mol Pharmacol. 1989 April; 35(4):469-76 and in Luthin G R and Wolfe B B. J Pharmacol Exp Ther. 228(3): 648, 1984. A typical example of such activation using contemplated compounds and compositions is presented in FIG. 4. Here, it should be particularly appreciated that M3 agonist activity was observed at remarkably low concentrations of contemplated compounds and compositions. For example, FC-03 (Green tea extract commercially available from VDF FutureCeuticals with at least 95% polyphenol content), FC-06 (Vitaberry plus extract commercially available from VDF FutureCeuticals with wild blueberry powder, grape seed extract powder, raspberry seed extract powder, cranberry powder, prune powder, tart cherry powder, wild bilberry powder, and silica dioxide), and FC-09 (Vitagrape extract commercially available from VDF FutureCeuticals with at least 90% total phenolic content) provided an at least 10% activation in the Sf9 assay over control at concentrations of less than 50 ppm, more typically less than 30 ppm, and most typically less than 20 ppm. Thirteen additional extracts comprising proanthocyanidins were tested and showed similar activity at the concentrations tested (data not shown). Therefore, it should be appreciated that in at least some aspects of the inventive subject matter, various oral care formulations may be provided in which the grape seed extract, proanthocyanidin, and/or plant extract (that is demonstrated to have M3 receptor agonist activity) has a concentration effective to achieve upon oral topical administration a concentration that is equal to or below 100 ppm, more typically equal to or below 50 ppm, and most typically equal to or below 25 ppm in the fluid that contacts the oral tissue (and particularly oral mucosal tissue).

Alternatively, simple in vivo tests measuring saliva flow can be performed following well-known methods (e.g., Ann Rheum Dis. 1992 April; 51(4):499-502). Thus, test results will be available from human (or other mammalian) subjects as well as from cell cultures. Thus, a test result may be obtained (by the provider of the composition or other party, including contract test laboratory) directly by in vivo tests on human subjects or by performing the M3 agonist activity test using in vitro tests, or indirectly by having the test(s) performed in a contract or otherwise affiliated laboratory, and even by having an independent and unaffiliated third party perform the test(s) and publish the test result. Especially contemplated test results will include those in which increase in saliva flow and/or M3 activation is reported as a function administration of the composition to a mammal (or other animal) or cells. Such report preferably provides qualitative information on the amount and/or type of the composition used and/or the increase in saliva flow and/or M3 activation achieved. Alternatively, or additionally, qualitative test results may also be provided that indicate in a generic manner that the composition is effective to increase salivary flow and/or volume, or effective to reduce a symptom associated with hyposalivation. Most typically (but not necessarily), the composition used for the test result has the same or similar ingredients than the composition that is marketed or otherwise provided to a consumer.

Thus, in especially preferred aspects of the inventive subject matter, the compositions contemplated herein will be provided to a consumer (typically the user) in association with the test result to inform or suggest to the consumer that the composition is effective to increase flow and/or volume of saliva and/or M3 activity. The term “in association with” is therefore meant to include any activity that logically (and most preferably also physically) couples the composition with the test result. For example, logical coupling includes displaying, printing, or otherwise providing information of the test result while making reference to the composition (e.g., displaying the test result and the composition). More preferably, however, the test result is physically associated with the composition. For example, such physical association may be performed by printing the test result on the container or packaging that holds the composition.

Of course, it should be appreciated that the above described exemplary formulations are merely illustrative with regard to the specific composition, and that numerous alternative compositions are also deemed suitable for use herein. For example, the proanthocyanidin may be provided as a relatively pure and isolated composition (e.g., purity greater than 50 mol %, more typically greater than 70 mol %, and most typically greater than 90 mol %), which may include a relatively complex mixture of chemically diverse polyphenols, or which may be produced by controlled oligomerization and/or polymerization of one or more species of polyphenolic compounds. Thus, contemplated proanthocyanidin compositions may be synthetic, or more preferably, isolated (e.g., as aqueous or alcoholic extracts) from one or more plant materials (typically fruit, seed, bark, or leaves), and especially from bilberry, cranberry, black currant, black tea leaves, and chokeberry. Consequently, it should be noted that the fruit/seed extract may be a multi-component extract that is more or less enriched in one or more components. Therefore, suitable extracts may include extracts prepared with one or more solvents, which may be further processed using one or more chromatographic steps.

With respect to the grape seed extract, it is generally preferably that the grape seed extract is commercially available (e.g., VDF FutureCeuticals), but also other extracts deemed suitable so long as such extracts provide an increase in saliva flow when used as described herein. For example, suitable alternative grape seed extracts are aqueous extracts, which may also involve use of a co-solvent (e.g., alcohols, ethyl acetate, etc.). While it is generally preferred that the solvent is at least partially removed from the extract to provide a solid or powder extract, appropriate extracts may also include at least a portion of the solvent and be therefore an aqueous or alcoholic solution.

On the other hand, as proanthocyanidins are commonly found in many plants and parts thereof (e.g., apple skin, pine bark, cinnamon bark, grape seed, grape skin, cocoa seed, bilberries, cranberries, black currants, choke berry, etc.), contemplated compositions may also include those where the proanthocyanidin is part of an extract from such plants or parts of the plant. As noted before, there are numerous manners of preparing plant extracts to enrich proanthocyanidins known in the art, and all of those are deemed suitable for use herein. Furthermore, it should be appreciated that the proanthocyanidins may also be present in already prepared compositions such as red wine, buckthorn oil, etc. Thus, all known proanthocyanidin/citrus extract-containing preparations are also deemed suitable for use herein.

As used herein, the term “proanthocyanidin” refers to an oligomeric or polymeric flavonol, which is typically found in fruits, bark, leaves, and seeds of various plants. It is noted that proanthocyanidins from leaves are generally less preferred, and in some aspects even excluded (e.g., leaves of Camellia sinensis). Most typically, proanthocyanidins will be a collection of chemically diverse oligomeric or polymeric flavonols. Still further, it is noted that resveratrol and oligomeric forms thereof are also considered a proanthocyanidin herein. Still further, it should be recognized that all of the proanthocyanidins contemplated herein may be further covalently bound to a glycosidic moiety (which may be a hexose, pentose, or even a disaccharide).

Typically, the quantity of the proanthocyanidins and/or grape seed extract will be at least 0.1-1 microgram per dose administered, more typically at least 10 microgram, and most typically at least 50 microgram. However, higher doses are also contemplated, especially where the proanthocyanidin is in a liquid formulation or controlled release formulation. Therefore, contemplated compositions may comprise between 0.01 wt % (and in some cases even less) and 10 wt % (and even higher) one or more proanthocyanidins and/or grape seed/plant extracts, especially where the composition is in liquid form or in a paste form and more typically between 0.1 wt % and 10 wt %. On the other hand, and especially where the composition is a solid composition (e.g., powder, dissolvable strip, lozenge), contemplated compositions may comprise between 1 wt % (and in some cases even less) and 75 wt % (and even higher) one or more proanthocyanidins and/or grape seed/plant extracts.

However, it should be noted that in preferred aspects of the inventive subject matter the proanthocyanidin (grape seed extract or plant extract) is present in relatively small quantities (typically less than 1 wt % of the administered composition) to achieve the desired effect. It should be particularly appreciated that the above findings are highly unexpected as proanthocyanidins and plant extracts comprising proanthocyanidins are known to have bitter taste and to produce an astringent sensation characterized as “drying”, “chalky”, or “adhesive” (e.g., Journal of the Science of Food and Agriculture, 2003, Volume 83 Issue 6, Pages 564-573), which is contrary to the effect observed at the concentrations presented herein. Thus, contemplated compounds and compositions will have an effective window in which the compounds and compositions will have a beneficial effect (increase in salivary production, flow and/or pH), below which no or only unsatisfactory effect is observed, and above which the beneficial effect turns into an undesirable effect. In most compositions and uses, the therapeutic or prophylactic window is between 0.1 mcg and 100 mcg per application, more typically between 1 mcg and 50 mcg per application, and most typically between 2 mcg and 20 mcg per application. Viewed from a different perspective, the therapeutic or prophylactic window in a composition (with respect to concentration of the proanthocyanidins and plant extracts comprising proanthocyanidins) will be between 0.01 wt % and 5 wt %, more typically between 0.1 wt % and 1 wt %, and most typically between 0.2 wt % and 0.5 wt %.

In still further preferred aspects, contemplated compositions will also include an organic acid, and most typically a nutritionally acceptable acid such as fruit acids (e.g., citric acid, malic acid, etc.) and/or an extract of a fruit, and particularly a citrus fruit. Especially suitable extracts include lemon and lime extracts, wherein the extract may be prepared from the whole fruit, the pulp, and/or rind (e.g., flavedo, and/or albedo). Depending on the type of fruit material, such extracts may be dehydrated and prepared in form of powders (e.g., from pulp or pressed juice), or as distillates (e.g., from rind) with variable essential oil content. Most preferably, the proanthocyanidin (or grape seed extract or M3 activating plant extract) composition and the acid/fruit extract are in a wt % ratio of between 1:100, more typically in a ratio between 1:5 to 1:50 and most typically in a ration between 1:10 to 1:30). However, other ratios are also deemed suitable and will typically depend on the particular type of formulation, desired flavor profile, etc. Additionally or alternatively, the organic fruit acid may also be provided as an isolated or synthetic compound.

Where desired, additional compounds can be added to the compositions contemplated herein, and particularly preferred compounds include those that increase palatability of the composition or formulation. Therefore, particularly preferred additional compounds include sweeteners (and especially artificial sweeteners) and flavoring agents (most preferably, essential oils or artificial flavors). The determination of particular amounts of these additional agents is well within the skill of the ordinary artisan and therefore not further elaborated on.

With respect to formulations comprising contemplated compounds and compositions, it is generally preferred that the compounds and compositions are formulated for topical oral administration, most preferably in a nutritionally or pharmaceutically acceptable carrier, most preferably in a presentation that allows a user to administer the composition in an outpatient or home setting. For example, suitable formulations include solid formulations such as free flowing powders (may be impregnated onto a carrier, including floss, Q-tip, and chewing gum), dissolvable strips, tablets, lozenges, etc., and liquids and gel formulations such as rinses, sprays, toothpaste, ointments, foams, etc. Moreover, suitable formulations may further include polymers for adhesion and sustained release, flavoring agents, thickeners, emulsifiers, humectants as known in the art of oral care products. Still further contemplated appropriate formulations are described in WO 2007/092811, which is incorporated by reference herein. Most preferably, contemplated compositions will be formulated to provide immediate effect and to sustain the effect over a period of at least 60 minutes, more typically at least 4 hours, and most typically at least 8 hours. Therefore, controlled release formulations are especially included herein. For example, and where desired, contemplated formulations may also be prepared as sustained release formulations, pharmaceutical preparation for local and/or systemic delivery, as pills, as a food additive to make dry/sticky food easier to eat/swallow, as a functional drink, and as artificial saliva extender, etc. In especially preferred alternative aspects, contemplated compositions may be combined with medication that is known to produce dry mouth. Such combination may be integrated into the formulation of the medication, or as supplement together with the formulation.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of providing relief from a symptom associated with hyposalivation, comprising:
formulating a topical composition for oral administration that includes at least one of a proanthocyanidin and a grape seed extract at a concentration effective to provide relief from the symptom;
obtaining a test result that indicates that the at least one of the proanthocyanidin and the grape seed extract provide relief from the symptom; and
providing the composition in association with the test result.

2. The method of claim 1 wherein the symptom associated with hyposalivation is selected from the group of dry mouth, bad breath, thickened saliva, altered sense of taste, and difficulty speaking or swallowing.

3. The method of claim 1 wherein the topical formulation is selected form the group consisting of a chewing gum, a dissolving strip, a tablet, a lozenge, a mouth rinse, a spray, a toothpaste, an ointment, a foam, a saliva extender, and a powder that is optionally impregnated on a carrier.

4. The method of claim 1 wherein the proanthocyanidin is incorporated into the topical formulation as part of an extract selected from the group consisting of a bilberry extract, a cranberry extract, a black currant extract, a black tea extract, and a chokeberry extract.

5. The method of claim 1 wherein the at least one of the proanthocyanidin and the grape seed extract are present at a concentration of equal or less than 1 wt % of the topical composition.

6. The method of claim 1 wherein the at least one of the proanthocyanidin and the grape seed extract are present at a concentration of equal or less than 0.1 wt % of the topical composition.

7. The method of claim 1 wherein the topical composition further comprises an organic acid or an extract from a citrus fruit.

8. The method of claim 7 wherein the extract from a citrus fruit is a citrus extract, and optionally wherein the citrus extract and the at least one of the proanthocyanidin and the grape seed extract are present in a synergistic combination.

9. The method of claim 1 wherein the test result is an test result that demonstrates in vitro M3 receptor agonist activity of the at least one of the proanthocyanidin and the grape seed extract or a test result that demonstrates in vivo an increase in salivary flow in a human.

10. An oral care product to provide oral comfort to a person affected by a symptom of hyposalivation, comprising a composition that includes at least one of a proanthocyanidin and a grape seed extract as sialagogue active ingredient at a concentration effective to alleviate a symptom associated with hyposalivation, and wherein the composition is formulated for oral topical administration.

11. The oral care product of claim 10 wherein the symptom associated with hyposalivation is selected from the group of dry mouth, bad breath, thickened saliva, altered sense of taste, and difficulty speaking or swallowing.

12. The oral care product of claim 10 wherein the topical formulation is selected form the group consisting of a chewing gum, a dissolving strip, a tablet, a lozenge, a mouth rinse, a spray, a toothpaste, an ointment, a foam, a saliva extender, and a powder that is optionally impregnated on a carrier.

13. The oral care product of claim 10 wherein the proanthocyanidin is incorporated into the composition as part of an extract selected from the group consisting of a bilberry extract, a cranberry extract, a black currant extract, a black tea extract, and a chokeberry extract

14. The oral care product of claim 10 wherein the at least one of the proanthocyanidin and the grape seed extract are present at a concentration of equal or less than 1 wt % of the topical composition.

15. The oral care product of claim 10 further comprising an organic acid or an extract from a citrus fruit.

16. An oral care product to provide oral comfort to a person affected by a symptom of hyposalivation, comprising a plant extract that is demonstrated to have M3 receptor agonist activity as a sialagogue active ingredient at a concentration effective to alleviate a symptom associated with hyposalivation, and wherein the composition is formulated for oral topical administration.

17. The oral care product of claim 16 wherein the symptom associated with hyposalivation is selected from the group of dry mouth, bad breath, thickened saliva, altered sense of taste, and difficulty speaking or swallowing

18. The oral care product of claim 16 wherein the topical formulation is selected form the group consisting of a chewing gum, a dissolving strip, a tablet, a lozenge, a mouth rinse, a spray, a toothpaste, an ointment, a foam, a saliva extender, and a powder that is optionally impregnated on a carrier.

19. The oral care product of claim 16 wherein the plant extract is selected from the group consisting of a bilberry extract, a cranberry extract, a black currant extract, a black tea extract, and a chokeberry extract.

20. The oral care product of claim 16 further comprising an organic acid or an extract from a citrus fruit.
